(19) United States
(12) Patent Application Publication
(10) Pub. No.: US 2010/0233243 A1
(43) Pub. Date: Sep. 16, 2010

(54) Laminated Microcapsule Sheet and Process For Production Thereof

(75) Inventors: Shinya Izumida, Shiga (JP); Takashi Iwade, Shiga (JP)

Correspondence Address: GLOBAL IP COUNSELORS, LLP, 1233 20TH STREET, NW, SUITE 700, WASHINGTON, DC, 20036-2680, US

(73) Assignee: TORAY ENGINEERING CO., LTD., Tokyo (JP)

(21) Appl. No.: 12/161,503

(22) PCT Filed: Jan. 18, 2007
(86) PCT No.: PCT/JP2007/050689

§ 371 Date: May 28, 2010

(30) Foreign Application Priority Data
Jan. 19, 2006 (JP) 2006-010750

(51) Int. Cl. A61K 47/00; B32B 37/14

(52) U.S. Cl. 424/439; 156/272.2

(57) ABSTRACT

Disclosed is a laminated microcapsule sheet which is produced using a lamination apparatus B and a water repellency imparting apparatus A, wherein the lamination apparatus B is used for laminating multiple types of materials at multiple parts on an edible film to form a laminated microcapsule sheet and the water repellency imparting apparatus A is used for imparting water repellency to the surface of the edible sheet. The sheet is improved in the disadvantages attributable to the water soluble property of an edible film.

### TECHNICAL FIELD

The present invention relates to a laminated microcapsule sheet and a process for production thereof. More particularly, the present invention relates to a laminated microcapsule sheet and a process for production thereof, which laminated microcapsule sheet is suitable for adhering to a intestinal mucosa so that drug is directly supplied to the intestinal mucosa.

### BACKGROUND ART

A conventional laminated microcapsule sheet is produced by adhering an enteric layer at multiple parts on an edible film using printing technique such as ink-jet technique and the like, each enteric layer having a diameter equal to or less than at least 1 mm, by adhering a drug layer on the enteric layer, and by adhering a barrier layer thereon which is not soluble in stomach nor bowel.

By employing the laminated microcapsule sheet produced by the above process, the microcapsule sheet adheres to the intestinal mucosa so that drug is directly supplied to the intestinal mucosa.

### DISCUSSION OF THE INVENTION

### Problems to be Solved by the Invention

A problem arises in that the drug layer cannot securely be adhered on the enteric layer due to the location of the enteric layer which is changed when the drug is adhered on the enteric layer because the edible film is dissolved its surface layer section so that the edible film is contracted when the enteric layer is adhered, due to the water solubility of the edible film.

Another problem arises in that microcapsules cannot be disposed with high density because the enteric layer is wetting and expanded on the edible film when the enteric layer is adhered with a predetermined thickness, due to the edible film having water solubility and the enteric layer being wetting and expanding when adhered on the edible film.

It is an object of the present invention to provide a laminated microcapsule sheet and a process for production thereof, which laminated microcapsule sheet solves the problems due to water solubility of an edible film.

### Means for Solving the Problems

A laminated microcapsule sheet according to the present invention comprises an edible film which has been imparted water repellency in its surface.

A process for production of a laminated microcapsule sheet according to the present invention comprises imparting water repellency to an edible film with plasma CVD (chemical vapor deposition). This process is a dry process so that disadvantage such as an edible film is solved by addition of water-repellent solution in the processing under wet environment, is prevented from occurrence, the disadvantage being observed in a wet process in which water-repellent solution including molecular fluorine is adhered to an edible film and the adhered water-repellent solution is dried.

Specifically, imparting water repellency is performed by supplying CF₄ gas under reduced pressure environment, and by doping molecular fluorine on the surface of the edible film by generating plasma in the surface side of the edible film.

Imparting water repellency is also performed by supplying SiH₄ gas (silane gas) and O₂ gas under reduced pressure environment, and by forming a SiO₂ membrane on a surface of an edible film by generating plasma in the surface side of the edible film. Imparting water repellency is also performed by supplying SiH₄ gas and NH₃ gas under reduced pressure environment, and by forming a SiN membrane on a surface of an edible film by generating plasma in the surface side of the edible film. Imparting water repellency is also performed by supplying SiH₄ gas, N₂O gas and N₂ gas under reduced pressure environment, and by forming a SiON membrane on a surface of an edible film by generating plasma in the surface side of the edible film.

### Effect of the Invention

The laminated microcapsule sheet according to the present invention has characteristic effects such that multiple types of material can securely be laminated by imparting water repellency to an edible film, and that microcapsules can be disposed with high density.

The process for production of a laminated microcapsule sheet according to the present invention has characteristic effects such that a disadvantage that an edible film is solved by water-repellent solution, is prevented from occurrence, and that water repellency is securely imparted.

### BRIEF DESCRIPTION OF THE DRAWINGS

FIG. 1 is a schematic view illustrating a water repellency imparting apparatus A in detail;

FIG. 2 is a schematic view illustrating a lamination apparatus B in detail;

FIG. 3 is a schematic plan view illustrating an example of a base member which is imparted water repellency for surrounding microcapsule forming face;

FIG. 4 is a schematic plan view illustrating another example of a base member which is imparted water repellency for surrounding microcapsule forming face; and

FIG. 5 is a schematic plan view illustrating another example of a base member which is imparted water repellency for surrounding microcapsule forming face.

### BEST MODE FOR CARRYING OUT THE INVENTION

Hereinafter, referring to the attached drawings, we explain embodiments of a laminated microcapsule sheet and a process for production of a laminated microcapsule sheet according to the present invention.

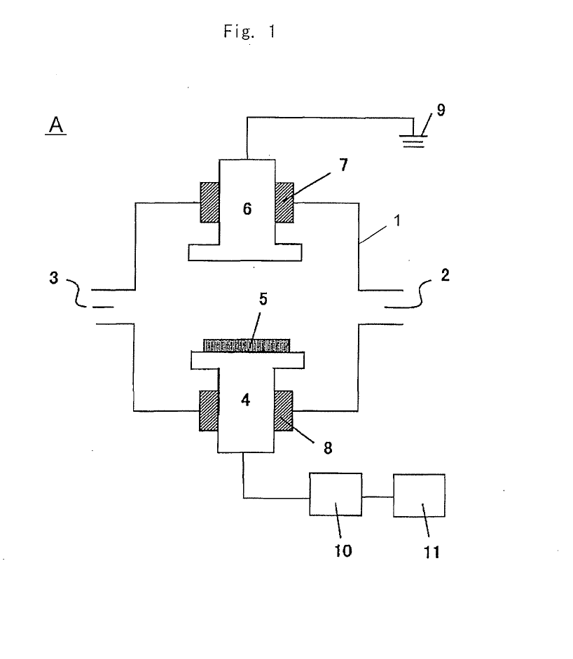

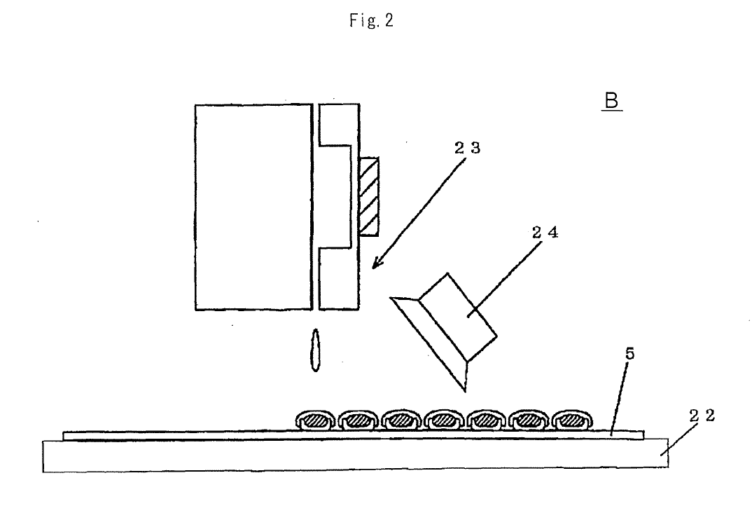

An embodiment of an apparatus for production of a laminated microcapsule sheet according to the present invention is schematically illustrated in FIGS. 1 and 2.

The apparatus for production of a laminated microcapsule sheet comprises a lamination apparatus B (refer to FIG. 2) for laminating multiple types of material on multiple parts on an edible film so as to produce a laminated microcapsule sheet, the lamination apparatus B being known from the past, and a water repellency imparting apparatus A (refer to FIG. 1) for imparting water repellency to a surface of the edible film.

FIG. 1 is a schematic view illustrating a water repellency imparting apparatus A in detail.

The water repellency imparting apparatus A is a capacity coupling type water repellency imparting apparatus. The water repellency imparting apparatus A comprises a vacuum vessel 1 having a gas feed port 3 and a vacuum exhaust outlet 2, and a plasma generating electrode 4 provided within the vacuum vessel 1. A partial part of the plasma generating electrode 4 is taken out from the vacuum vessel 1 through the intermediary of an insulating member 8, the insulating member 8 doubling as a vacuum seal and an electric insulator, and is connected to an output side of a high frequency impedance matching circuitry (matcher) 10. A high frequency power supply 11 is connected to the input side of the matcher 10 through a coaxial cable. By such arrangement, the plasma generating electrode 4 is supplied high frequency power. The plasma generating electrode 4 may be an electrode made of various shapes, such as an electrode made of stainless steel pipe, an electrode made of plane plate, or the like. Further, an opposition electrode 6 is provided on almost the central axis of the plasma generating electrode 4. The opposition electrode 6 acts as an edible film fixing jig. The edible film fixing jig 6 may be made of stainless steel, for example. The edible film fixing jig 6 that is also made of stainless steel for example, is taken out its partial part from the vacuum vessel 1 through the intermediary of an insulating member 7, similarly to that of the plasma generating electrode 4, the insulating member 7 doubling as a vacuum seal and an electric insulator, and is connected to a ground 9. An edible film 5 is set on the plasma generating electrode 4.

Then, an example of imparting water repellency to an edible film 5 is described.

At first, the vacuum vessel 1 is discharged its air to reach vacuum condition of 10⁻³ Pa. Then, CF₄ is flowed as processing gas from the gas feed port 3. Other gas than CF₄ may be employed as the processing gas, as long as gas can generate fluorinated ion within plasma. And, pressure within the vacuum vessel 1 is kept to be 50 Pa, and the high frequency power supply 11 is turned on so that the high frequency power of 300 W is supplied to the plasma generating electrode 4. At this time, a variable condenser of the matcher 10 is adjusted so as to minimize high frequency reflected power.

An edible film 5 imparted water repellency to its surface, is obtained by carrying out the water repellency imparting processing for 1 minute.

When water is dropped on a processed face of the edible film 5 which is imparted water repellency to its surface, and when 3 minutes has passed, the edible film 5 is not dissolved and is not observed any change.

But, when the edible film 5, which is imparted water repellency to its surface, is immersed entirely in water, the edible film 5 is dissolved immediately.

Therefore, the surface imparted water repellency effectively acts when a laminated microcapsule sheet is produced, while, after the production, the laminated microcapsule sheet adheres intestinal mucosa so as to directly administer drug to the intestinal mucosa by dosing.

FIG. 2 is a schematic diagram illustrating a lamination apparatus B in detail.

The lamination apparatus B comprises, for example, an XY stage 22 for supporting an edible film 5 which has imparted water repellency in its surface and for moving the edible film 5 two dimensionally, a nozzle device 23 having three liquid drop supplying nozzles for supplying a liquid drop for enteric layer, a liquid drop for drug layer, and a liquid drop for barrier layer which is not dissolved in stomach nor in bowel, respectively, from above the edible film 5, so as to adhere on the surface of the edible film 5, and a drying device 24 for drying the adhered liquid drops.

The liquid drop supplying nozzle discharges a liquid drop using a piezo head, the piezo head discharging a liquid drop utilizing distorting characteristic of piezo ceramics, or a thermal ink jet head, the thermal ink jet head discharging a liquid drop utilizing thermal energy, for example, similarly to an ink jet nozzle used in an ink jet printer, so that discharging amount is controlled with high accuracy.

The nozzle device 23 has three liquid drop supplying nozzles, so that the nozzle device 23 is arranged to move to and fro corresponding to the placement of the three liquid drop supplying nozzles for sequentially operating the three liquid drop supplying nozzles at the same location.

An infrared heater, for example, is employed as the drying device 24. When the device for producing a laminated microcapsule sheet having the above arrangement is employed, at first, a surface of an edible film is imparted water repellency by the water repellency imparting apparatus A, then, an enteric layer, a drug layer, and a barrier layer are laminated on the surface of the edible film 5 by supplying the edible film 5 having imparted water repellency to the lamination apparatus B, so that a laminated microcapsule sheet is obtained.

In this case, laminating of the enteric layer, the drug layer, and the barrier layer is carried out on the surface of the edible film having imparted water repellency. Therefore, disadvantage that a surface layer of an edible film is dissolved so that a location of a laminated layers is changed, is prevented from occurrence. As a result, an accurate laminated arrangement is obtained.

Further, laminated microcapsules are disposed at high density, because the surface layer of the edible film is not wetted and expanded.

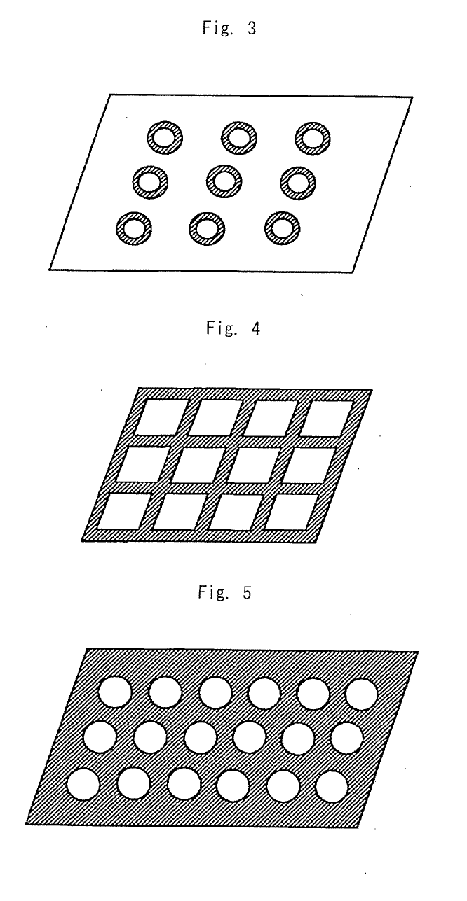

When water repellency is imparted to an edible film 5 using plasma CVD (chemical Vapor Deposition), it is preferable that water-repellent frames (ring shaped frames, lattice shaped frames and the like) (refer to dashed portion in FIGS. 3 and 4) are patterned around microcapsule forming faces, as is illustrated in FIGS. 3 and 4, using a mask having a predetermined pattern. Thus, the enteric layer is prevented wetting and spreading from occurrence, so that material for enteric layer exists within the water-repellent pattern. Consequently, a laminated microcapsule can be miniaturized, and can be disposed with high density, by determining the water-repellent pattern to be smaller.

Further, it is also possible that water repellency processing is applied to entire surface of an edible film, then UV (ultra violet) radiation is applied through opening sections of a predetermined mask, so that faces corresponding to the opening sections (corresponding to microcapsule forming faces) lose water repellency and become hydrophilic, and faces other than the microcapsule forming faces (refer to shaded sections in FIG. 5) are water repellency, as is illustrated in FIG. 5. Thus, the enteric layer is prevented wetting and spreading from occurrence on a microcapsule forming face, so that a laminated microcapsule having a small diameter can be formed. In this case, the enteric layer is prevented wetting and spreading from occurrence, so that material for enteric layer exists within the water repellent pattern. Consequently, by miniaturizing the water repellent pattern, the laminated microcapsules can be miniaturized, and are disposed at high density.

What is claimed is:

1. A laminated microcapsule sheet made by laminating multiple types of material at multiple parts on an edible film, is characterized in that water repellency is imparted to a surface of the edible film.

2. A process for production of a laminated microcapsule sheet which is made by laminating multiple types of material at multiple parts on an edible film, is characterized in that imparting water repellency is carried out for the edible film using plasma CVD.
